(19) United States
(12) Patent Application Publication
(10) Pub. No.: US 2011/0144416 A1
(43) Pub. Date: Jun. 16, 2011

(54) INFANT SLEEPING APPARATUS

(76) Inventor: Joshua Waddell, Hays, KS (US)

(21) Appl. No.: 12/635,812

(22) Filed: Dec. 11, 2009

(51) Int. Cl. A61M 21/02; A47D 7/00

(52) U.S. Cl. 600/26; 5/93.1

(57) ABSTRACT

An apparatus that simulates the movement an infant might experience while being held on its mother's chest is provided. The apparatus produces controlled and rhythmic motions similar to the expansion and contraction of an adult's chest or torso during normal breathing. The apparatus, and methods of operation thereof, provide a soothing, sleep-inducing motion for the infant placed therein. The apparatus may also include structure for causing circulation of air around the apparatus so as to prevent the build-up of carbon dioxide near the infant.

### BACKGROUND OF THE INVENTION

1. Field of the Invention

The present invention generally pertains to an infant sleeping apparatus that generates a motion which mimics the breathing patterns of the mother. In certain embodiments, the apparatus includes structure for creating an airflow about the apparatus to maintain optimal temperatures and remove carbon dioxide gases to promote a healthy, positive sleeping environment for newborns and infants.

2. Description of the Prior Art

Calming a crying newborn, and particularly so that the child will fall asleep, can be a significant and stressful challenge to the parents of the child. Research has shown that the deprivation of the maternal biological rhythms can be a cause of irregular behavior and sleeping patterns in pre-term infants. Also, the rhythmic stimulation provided in utero has been shown to enhances neurological maturation, which in turn facilitates the development of state behavior organization. A number of devices exist that mimic the sound of a mother's heartbeat or use rhythmic-type motion to calm a child and induce sleep. However, the rhythmic motion devices generally produce a rocking-type motion that does not very well approximate the natural bodily motions of the mother.

Respiratory health of the child is also a point of concern for newborns and infants. Sudden infant death syndrome (SIDS) is a syndrome marked by the sudden, unexpected death of an infant that remains unexplained after a thorough forensic autopsy. According to a study published in October 2007 in the Journal of the American Medical Association, babies who die of SIDS have abnormalities in the brain stem (the medulla oblongata), which helps control functions like breathing, blood pressure and arousal. Sleeping on the back has been recommended as a way of avoiding SIDS. It is theorized that small infants with little or no control of their heads may, while lying face down, inhale their exhaled breath (high in carbon dioxide) or smother themselves on their bedding. Further, additional research suggests that babies with a particular genetic makeup do not react “normally” by moving away from the pooled CO₂, and thus smother. Yet another theory supporting the recommendation to place babies on their backs to sleep is that babies sleep more soundly when placed on their stomachs and are unable to rouse themselves when they have an incidence of sleep apnea, which is thought to be common in infants.

In an effort to promote good airflow, many cribs and bassinets are designed with mesh or netting around the sleeping area. However, these measures are not directed toward actively drawing carbon dioxide gas away from the infant while sleeping.

### SUMMARY OF THE INVENTION

According to one embodiment of the present invention there is provided an apparatus into which an infant may be placed and operable to simulate the respiratory patterns of an adult. The apparatus comprises a bed structure into which the infant may be placed and a base assembly including a mechanism for powered raising and lowering of the bed structure between a first lowered position and a second raised position. The mechanism operates to maintain the bed structure at substantially the same level of inclination during shifting between the first and second positions.

In another embodiment of the present invention there is provided an infant-supporting apparatus comprising a base assembly and a bed unit received on the base assembly and configured to receive an infant therein. The base assembly generally comprises a platform pivotally connected to a stationary support through one or more linkage members, a rotatable cam operably coupled to at least one of the linkage members, and a motor operably coupled with the cam for powered rotation thereof. The motor, cam, and linkage members are operable to effect shifting of the platform between a first lowered position and a second raised position while maintaining the platform at a generally constant incline throughout the shifting thereof. The bed unit is raised and lowered along with the platform during shifting of the platform between the first and second positions.

In yet another embodiment of the present invention there is provided a method of providing therapeutic motion for an infant. The method first comprises the step of placing an infant in an infant-supporting apparatus comprising a mechanism for powered shifting of the apparatus between a first lowered position and a second raised position. Next, the mechanism is activated to cause the apparatus to oscillate between the first lowered position and the second raised position, wherein the time in which the apparatus shifts from the first lowered position to the second raised position is less than the time in which the apparatus shifts from the second raised position back to the first lowered position.

### BRIEF DESCRIPTION OF THE DRAWINGS

FIG. 1 is a perspective view of an infant sleeping apparatus in accordance with one embodiment of the present invention;

FIG. 2 is an expanded top perspective view of the infant sleeping apparatus;

FIG. 3 is an expanded bottom perspective view of the infant sleeping apparatus;

FIG. 4 is a sectioned, perspective view of the infant sleeping apparatus;

FIG. 5 is a cross-sectional view of the infant sleeping apparatus in its lowest configuration at the beginning of a raising and lowering cycle;

FIG. 6 is a cross-sectional view of the infant sleeping apparatus in its highest point in the raising and lowering cycle;

FIG. 7 is a cross-sectional view of the infant sleeping apparatus in an intermediate position subsequent to the highest point and near the lowest point in the raising and lowering cycle;

FIG. 8 is a cross-sectional view of the infant sleeping apparatus once again in its lowest configuration near the end of the raising and lowering cycle;

FIG. 9 is an side elevation view of a the cam assembly of the infant sleeping apparatus containing a specially configured cam and depicting various segments of its rotational travel during a raising and lowering cycle of the apparatus;

FIGS. 10a-d are side elevation views of the cam of FIG. 9 in various rotational stages and depicting the shifting of the platform caused by rotation of the cam; and

FIG. 11 is a graph illustrating the change in platform elevation during rotation of the cam through one raising and lowering cycle.

### DETAILED DESCRIPTION OF THE PREFERRED EMBODIMENT

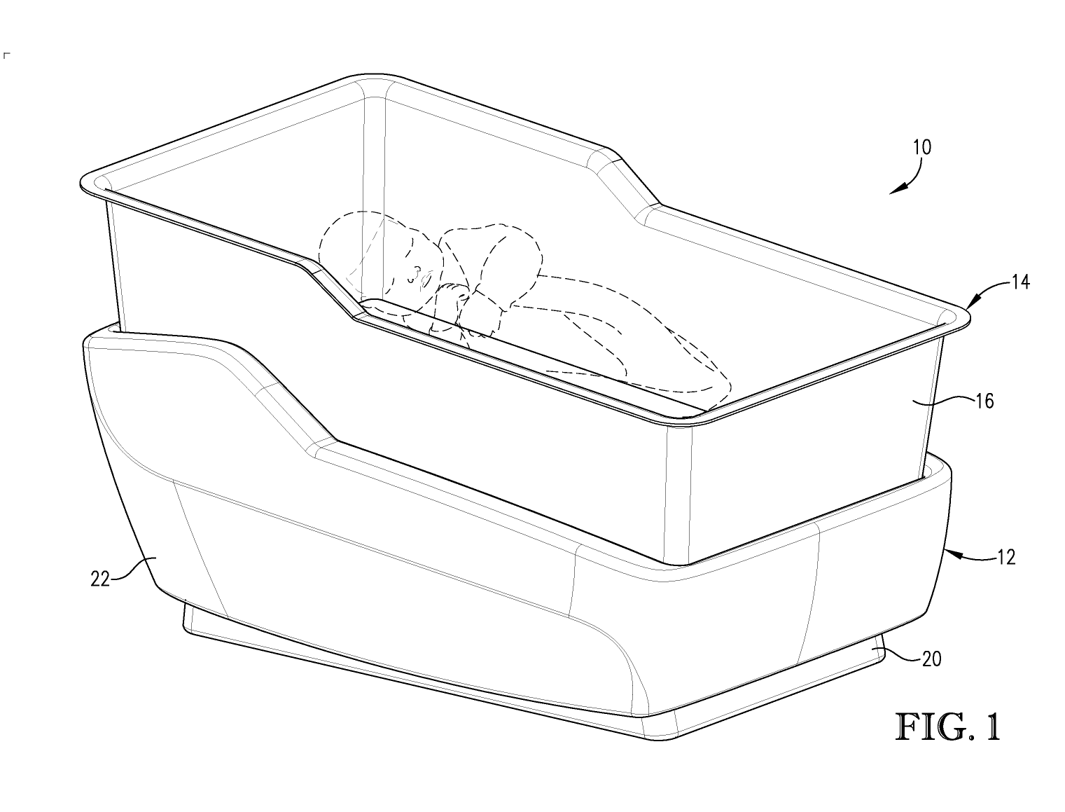

FIG. 1 depicts an infant sleeping apparatus 10 in accordance with one embodiment of the present invention. Apparatus 10 is configured to receive an infant and provide stable, vertical motion that mimics the rhythmic breathing patterns of a mother who might be holding the child on her chest. By simulating such motion, the apparatus provides the benefit of calming the infant so as to enable the infant to fall asleep.

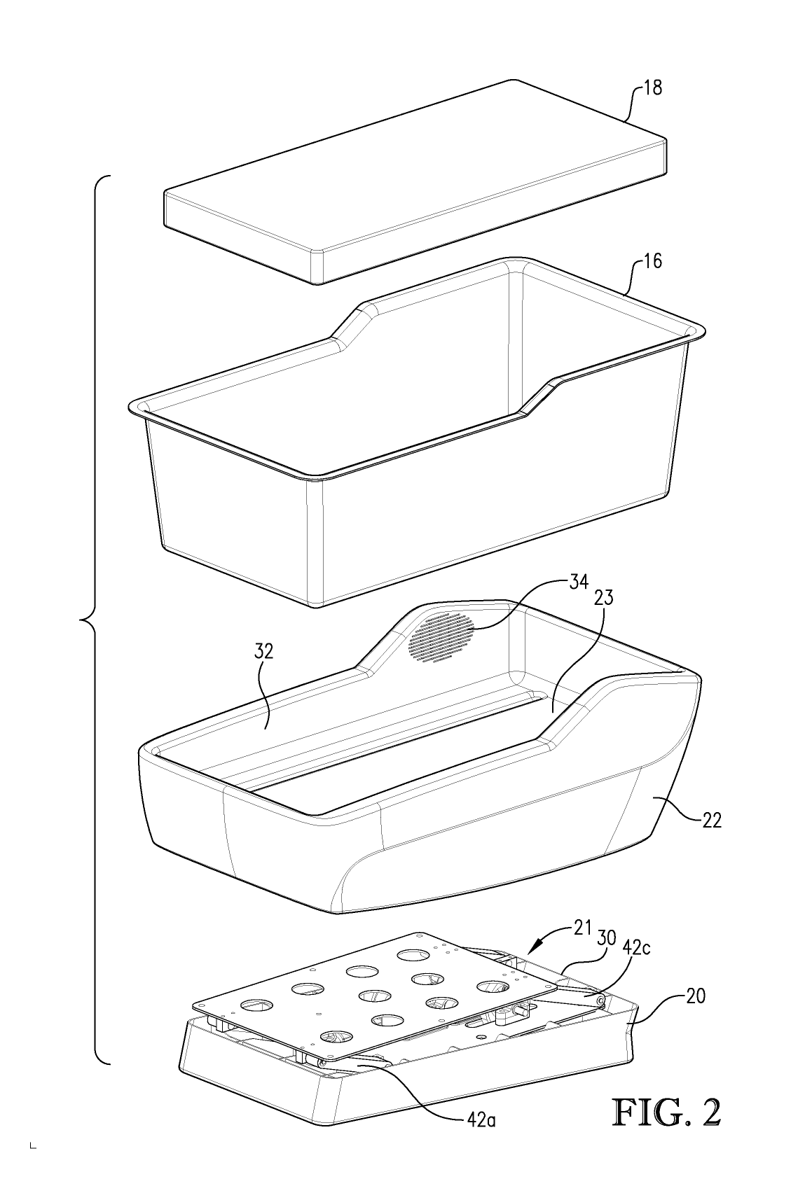

Generally, apparatus 10 comprises a base assembly 12 and a bed unit 14. Bed unit 14 is received on base assembly 12 and configured to receive an infant laid therein. In certain embodiments, bed unit 14 comprises a bassinet 16, such as those commonly used in hospital maternity wards having a footprint of 26.5″×14″. In additional embodiments, apparatus 10 is capable of accommodating an infant approximately 27″ long and weighing approximately 17 lbs. Bed unit 14 also comprises an optional sleeping pad 18 (see, FIG. 2), especially a hypo-allergenic, water resistant, flame retardant, nonconductive sleeping pad which fits “loosely” in the bassinet for easy removal. Alternatively bed unit 14 may comprise a hospital isolette unit, such as typically used with premature infants requiring intensive care.

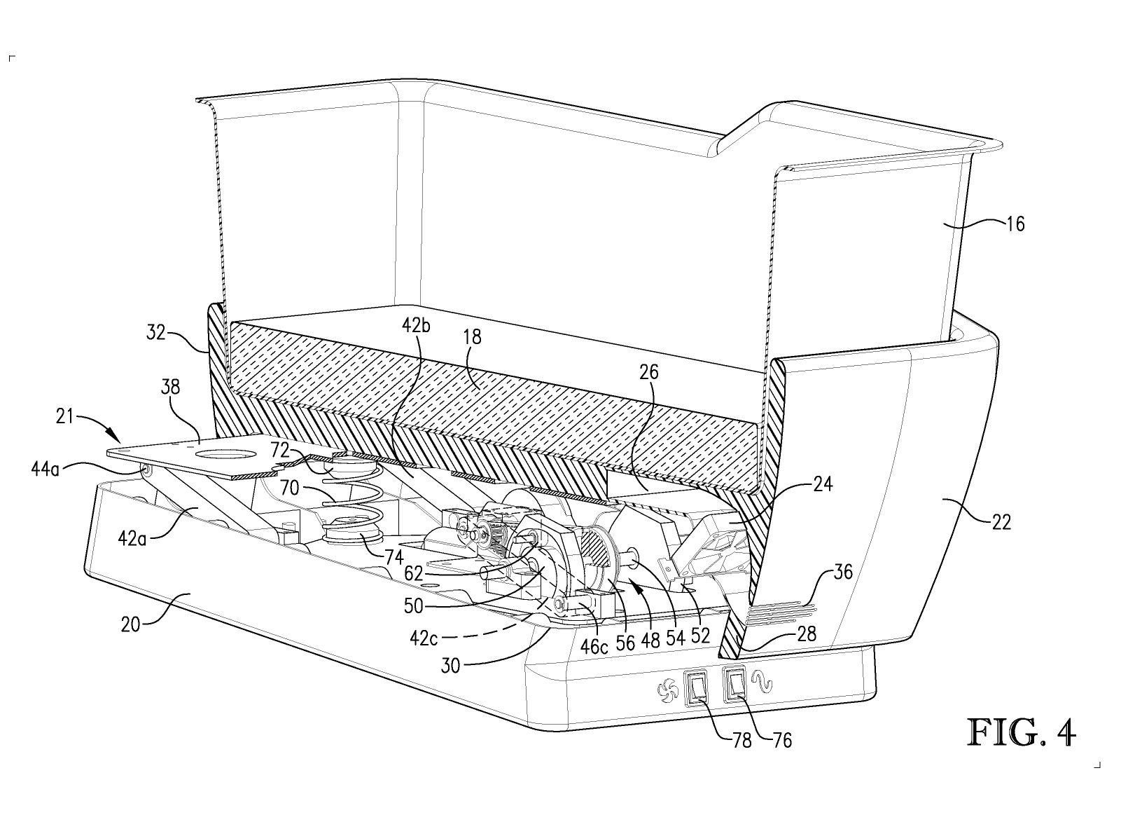

Base assembly 12 generally comprises a lower housing 20 containing a mechanism 21 for effecting controlled raising and lowering of bed unit 14 and an upper portion 22 that is mounted on the lower portion and optionally includes an electrical fan 24 (see, FIG. 4) disposed within duct 26 and described in further detail below. In one particular embodiment according to the present invention, lower housing 20 has dimensions of 24″×12″×3″, and upper portion has dimensions of 29″×16.5″×10″. As best seen in FIG. 4, upper portion 22 includes a circumscribing skirt region 28 that extends below the upper most edge 30 of housing 20. Skirt region 28 shields edge 30 during the raising and lower cycle of bed unit 14 so as to prevent foreign matter from entering housing 20 that could damage mechanism 21 or simply create an unsanitary condition within the housing.

It is also within the scope of the present invention for upper portion 22 to function as the bed unit, so as to eliminate the necessity for bassinet 16. For example, upper portion 22 includes a circumscribing raised sidewall portion 32 into which a pad may be placed and the infant laid thereupon. Sidewall portion 32 would operate to keep the infant contained within apparatus 10 during use. However, it is appreciated that the use of a separate bassinet 16 may have certain advantages, particularly in that bassinet 16 may be detached for easier cleaning and can be moved from a cart or other carrier device to apparatus 10 and back.

As noted above, upper portion optionally includes air-handling structure comprising fan 24 located within duct 26 operable to circulate air around apparatus 10. The build up of carbon dioxide levels near a sleeping infant has been identified as a possible factor of SIDS. The air handling structure is directed toward preventing carbon dioxide from accumulating around apparatus 10 so as to alleviate this concern. Fan 24 operates to pull air from the top side of upper portion 22 through at least one vent 34, and preferably two vents (one on each side), disposed in sidewall portion 32 near the end where the infant's head would normally be placed. The air travels into duct 26, through fan 24, and is discharged through an outlet 36 located in or near skirt region 28. It is within the scope of the present invention to employ other means of air movement, including a passive air flow system based on the inherent movement of the platforms, a linear actuator, pneumatic cylinder, pneumatic bellows or an air bladder that would imitate the natural sounds and feelings of the human lung.

Mechanism 21 generally comprises a movable platform 38 coupled with a stationary support 40 via a plurality of linkage members 42a-d. Each linkage member 42 is pivotally connected to both platform 38 and support 40 at joints 44a-d, 46a-d, respectively. Linkage members 42c and 42d are operably coupled with a cam assembly 48 comprising cams 50, 52 mounted on an axle 54. Also mounted on axle 54 is a drive wheel 56 that is coupled to an electric motor 58 via drive belt 60. Upon actuation of motor 58, belt 60 turns drive wheel 56 which in turn rotates axle 54 and cams 50, 52. Motor 58 also provides some light background noise which can be soothing to the infant.

Linkage members 42c and 42d are provided with cam follower bearings 62 disposed within a cam groove 64. Groove 64 is defined by a raised outer cam sidewall 66 and a raised inner hub 68. As cams 50, 52 are rotated, followers 62 slide or roll in groove 64, the vertical movement of followers 62 being restricted by sidewall 66 and hub 68. As can be seen in the Figures, cams 50, 52 are specially configured so that as followers 62 travel within groove 64, linkage members 42c and 42d are shifted upward and downward thereby causing platform 38 to raise and lower with respect to stationary support 40. The motion caused by rotation of cams 50, 52 is described in further detail below.

An optional spring 70 may be used to reduce stress upon motor 58 during the raising and lowering of platform 38. Spring 70, as shown, is a coil spring that when installed within apparatus 10 is under compression. The ends of spring 70 are coiled about post 72 attached to platform 38 and post 74 attached to support 40. As platform 38 is raised, spring 70 assists in forcing platform 38 and support 40 apart. When platform 38 is being lowered, spring 70 cushions platform 38 thereby helping to avoid jarring shifting thereof. As illustrated, linkage members 42a and 42b are not operably coupled with the cam assembly. Instead, linkage members 42a and 42b merely follow along with the shifting of platform 38 as a result of the movement of linkage members 42c and 42d that is induced by rotation of cams 50, 52. Pivot joints 44a, b and 46a, b may be provided with detents to prevent pivoting of linkage members 42a and 42b beyond predetermined limits.

Switches 76, 78 located in housing 20 control the operation of motor 58 and fan 24, respectively. As shown, the switches are toggle on/off switches; however, dial or multiple setting switches could be employed if finer control over motor speed or fan speed was desired. Switch 76 also allows for the operator to selectively turn off motor 58 so as to wean the infant off of the motion of apparatus 10 while still permitting fan 24 to operate.

It is understood that mechanism 21 shown in the Figures and described above is exemplary and should not be taken as limiting the scope of the present invention. Other means for effecting raising and lowering of the apparatus may be used such as an expandable air bladder, pneumatic or hydraulic cylinders, so long as the mechanism employed is operable to achieve motion similar to the breathing patterns of an adult.

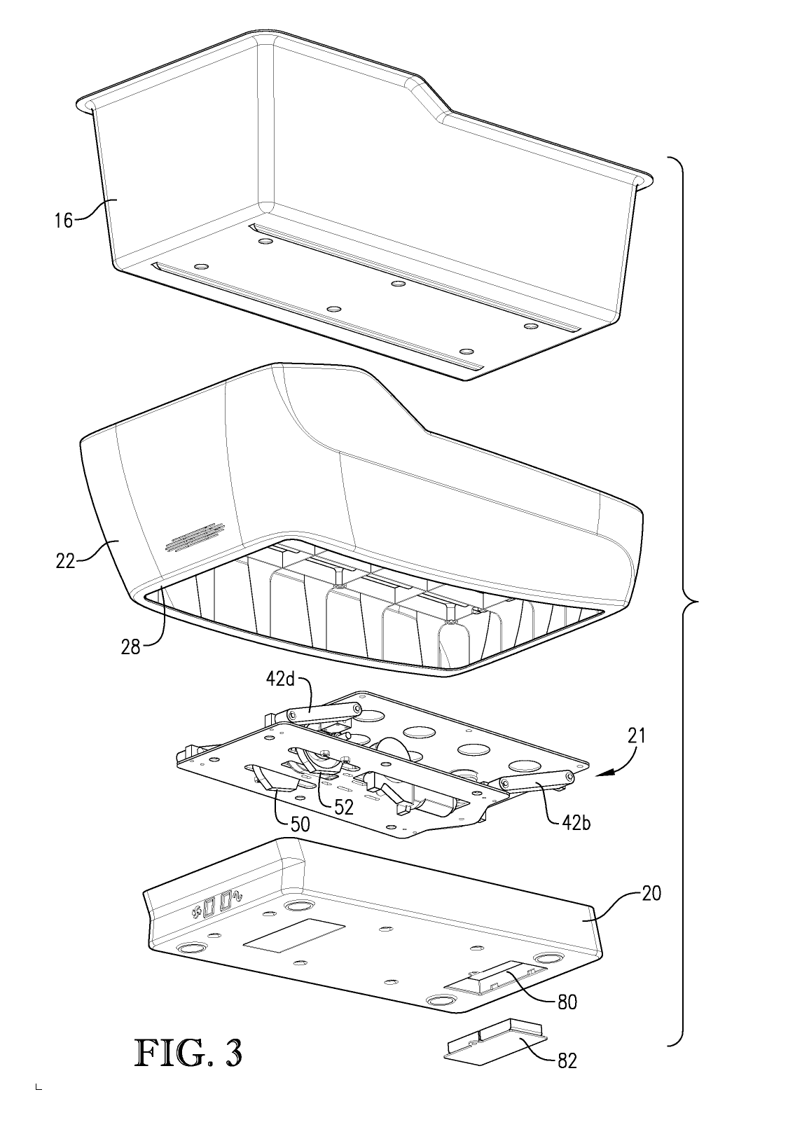

Apparatus 10 may be configured to run off of DC battery power or 110 volt AC power. In certain embodiments, it may desirable to employ battery power in that the apparatus 10 becomes entirely self-contained, is easily portable, and risk of electrical shock to the infant is substantially reduced if not entirely eliminated. In these embodiments, as can be seen in FIG. 3, housing 20 is also provided with a battery compartment 80 and cover 82. In alternate embodiments, apparatus 10 may be provided with a standard electrical plug (and associated circuitry) in addition to battery compartment 80 so that the user may be able to choose to operate apparatus 10 off of either battery or AC power.

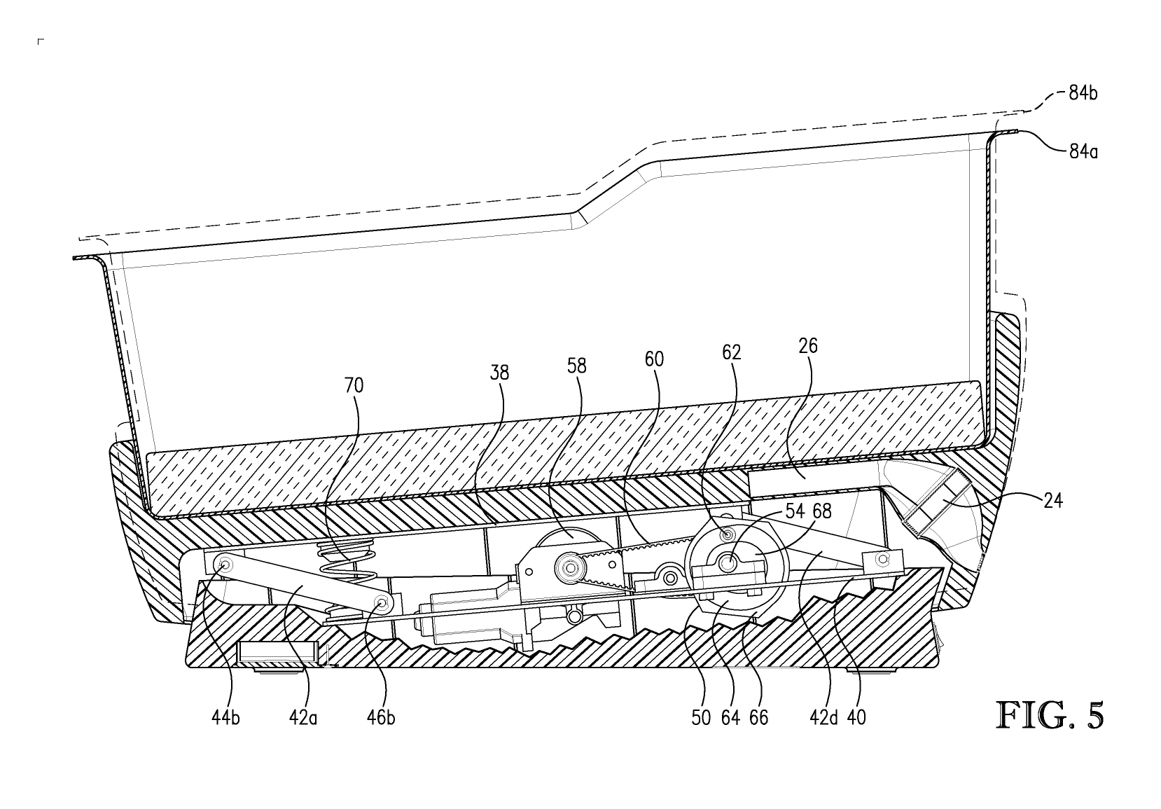

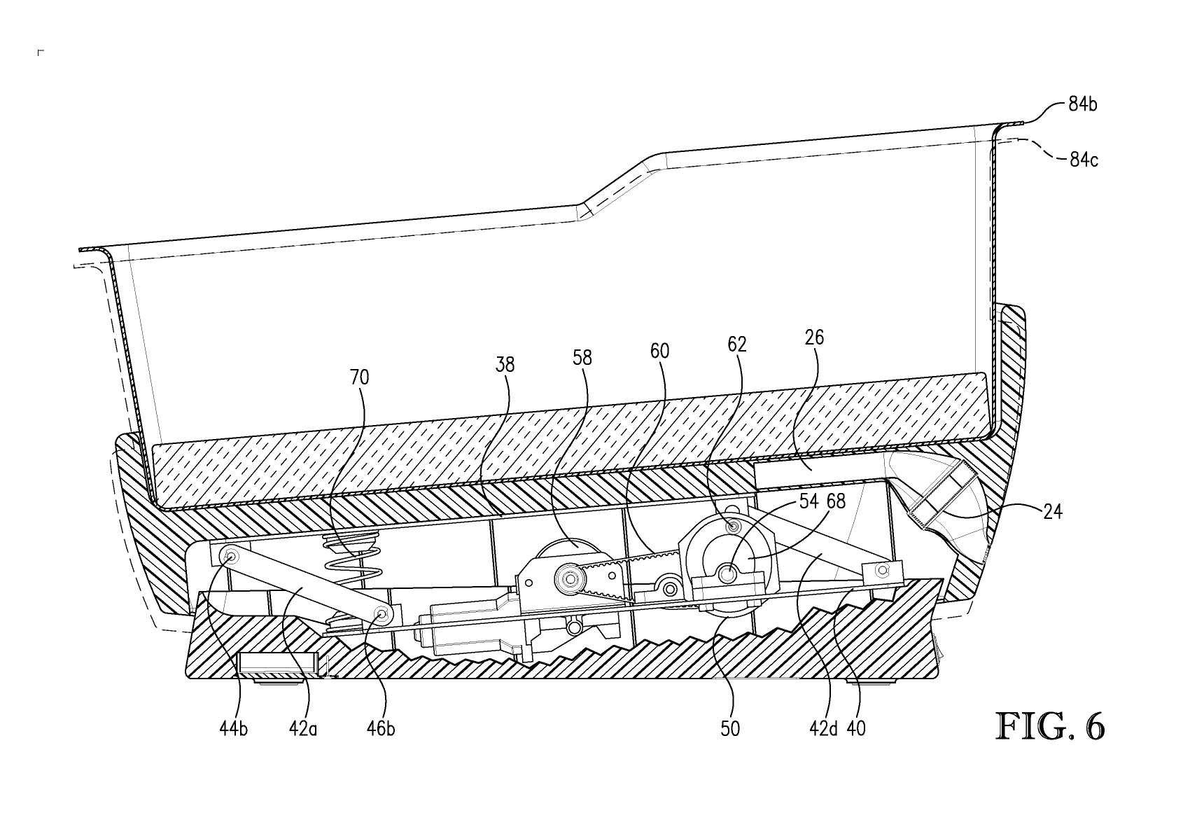

FIGS. 5-8 depict apparatus 10 in various stages throughout the raising and lowering cycle of platform 38 and base upper portion 22. FIG. 5 illustrates the beginning of a raising and lowering cycle in which platform 38 is in its lowest position relative to stationary support 40. Note, cam hub 68 is not a concentric circle, but oblong and “egg-shaped” with axle 54 being skewed toward the end of hub 68 having the smallest radius of curvature. Note the position of the bassinet upper margin 84a and 84b, shown in phantom. Margin 84b represents the height of the bassinet upper margin upon shifting to the highest point of the raising and lowering cycle as shown in FIG. 6.

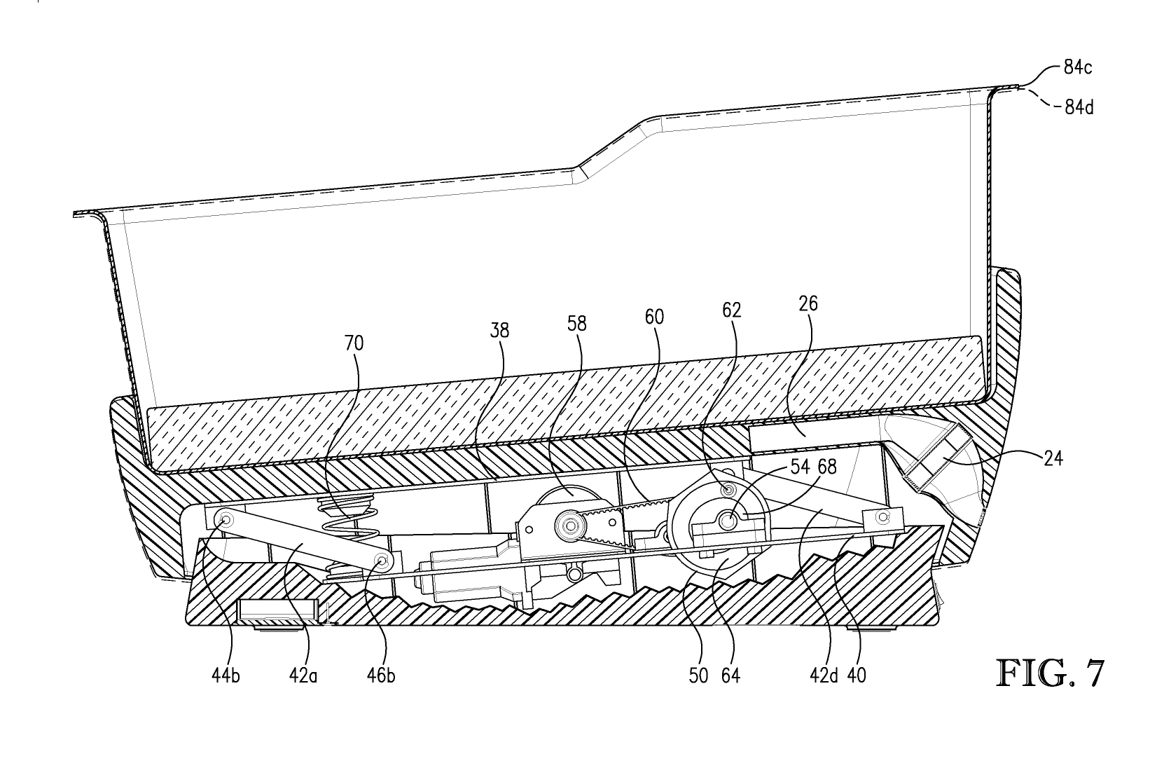

As shown in FIG. 6, cam 50 has been rotated counterclockwise so that follower 62 is at its farthest point from axle 54, i.e., the linear distance between follower 62 and axle 54 is at its maximum point. Thus, follower 62 and linkage members 42a-d have been pivoted upward thereby raising platform 38 and base upper portion 22. Bassinet upper margin 84b is shown at its highest elevation during the raising and lowering cycle. Margin 84c, shown in phantom, represents the configuration in the raising and lowering cycle depicted in FIG. 7, one in which the bassinet upper margin is approaching the lowest point.

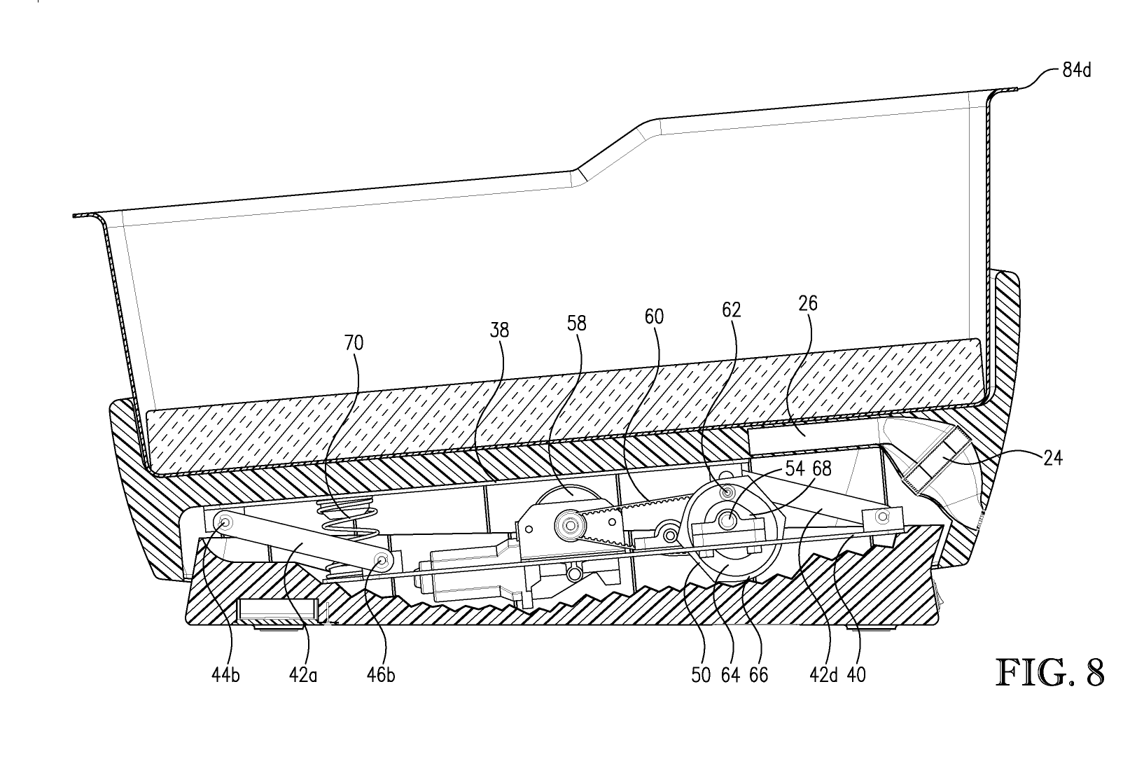

FIG. 7 depicts cam 50 approaching the lowest position in the raising and lowering cycle, as indicated by margin 84c and margin 84d, shown in phantom. Cam 50 has been rotated counterclockwise so that follower 62 is approaching its closest point to axle 54. The shape of cam hub 64 is such that from this point forward the radial distance between axle 54 and follower 62 will only slightly change as cam 50 continues to rotate, thus explaining why the bassinet upper margin shifts very little between that shown in FIGS. 7 and 8. As shown in FIG. 8, margin 84d has returned to substantially its lowest elevation in the raising and lowering cycle. Margin 84d is essentially at the same height as margin 84a shown in FIG. 5; however, it is plainly visible that cam 50 has yet to complete a full revolution. As cam 50 continues its counterclockwise rotation back to the start of the raising and lowering cycle of FIG. 5, the bassinet upper margin remains essentially fixed in position. This very slight change produces a “pause” effect simulating the natural pause between breaths in an adult's respiratory cycle.

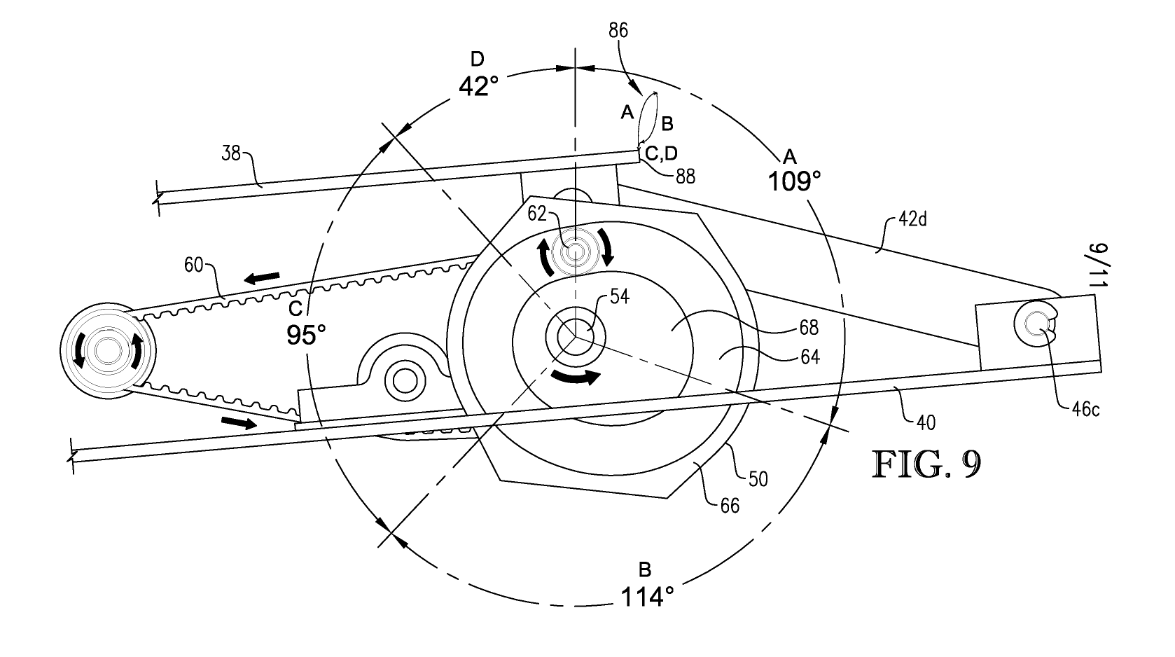

FIG. 9 illustrates the approximate position on cam 50 for each of the configurations shown in FIGS. 5-8. In FIG. 9 cam 50 is shown in the same position as in FIG. 5, depicting the beginning of a raising and lowering cycle. In order to reach the configuration shown in FIG. 6, cam 50 must rotate counterclockwise through approximately 109 degrees. This rotational segment is identified as part “A” of the raising and lowering cycle. Next, the cam rotates counterclockwise through approximately another 114 degrees to reach the configuration shown in FIG. 7. This rotational segment is identified as part “B” of the raising and lowering cycle. Following part B, the rate of change in the elevation of platform 38, and consequently the upper margin of the bassinet, slows considerably. Cam 50 continues to rotate counterclockwise through approximately another 95 degrees to reach the configuration shown in FIG. 8. This rotational segment is identified as part “C” of the raising and lowering cycle. The elevation of platform 38 remains essentially constant over approximately the next 42 degrees of rotation, identified as part “D” of the raising and lowering cycle, until cam 50 arrives back at its starting position.

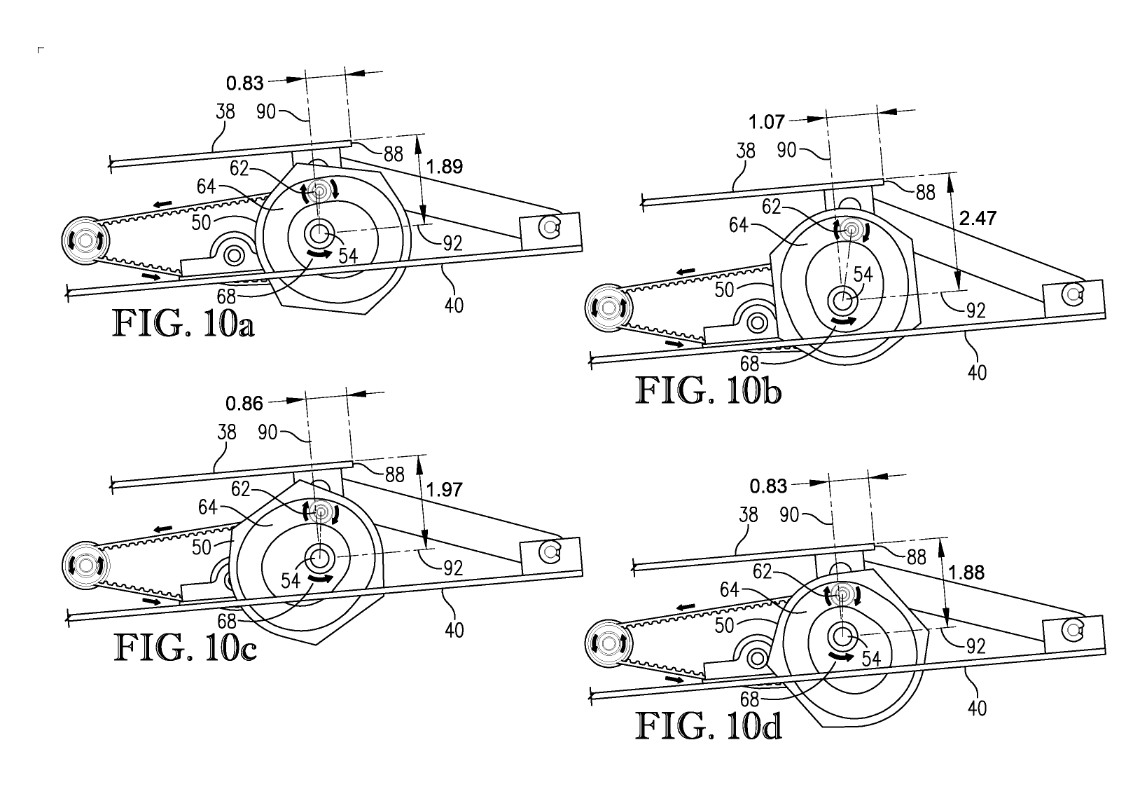

FIG. 9 also contains a series of smaller arrows 86 that depict the travel of platform edge 88 during the various parts of the raising and lowering cycle. As can be seen, this travel is primarily vertical, but also contains a slightly horizontal component as well. This path of travel of platform 38 is illustrated in terms of absolute distances in FIGS. 10a-d. Note that these measurements are merely illustrative and should not be taken as limiting the scope of the present invention.

FIG. 10a shows cam 50 in the same configuration as in FIG. 5, at the beginning of the raising and lowering cycle. As can be seen, the distance between an axis 90 through axle 54 and perpendicular to platform 38 is 0.83 inches from edge 88. Also, an axis 92 through axle 54 and parallel to platform 38 is 1.89 inches from the top margin of edge 88. FIG. 10b shows cam 50 in the same configuration as in FIG. 6. Note, though that the distance between axis 90 and edge 88 has increased to 1.07 inches indicating roughly one-quarter inch of horizontal shifting. While at the same time, platform 38 has been elevated by just over one-half inch. FIG. 10c shows cam 50 in the same configuration as in FIG. 7. Note that the distance between axis 90 and edge 88 is nearly back to the original mark, while the elevation of platform 38 is still elevated nearly a tenth of an inch from its original position. FIG. 10d shows cam 50 in the same configuration as in FIG. 8. Note that the distance between axis 90 and edge 88 is substantially the same as in FIG. 10a, as is the elevation of platform 38. Therefore, it can be appreciated that during shifting from the cam position of FIG. 10d back to the starting point of FIG. 10a, the position of platform 38 remains essentially fixed.

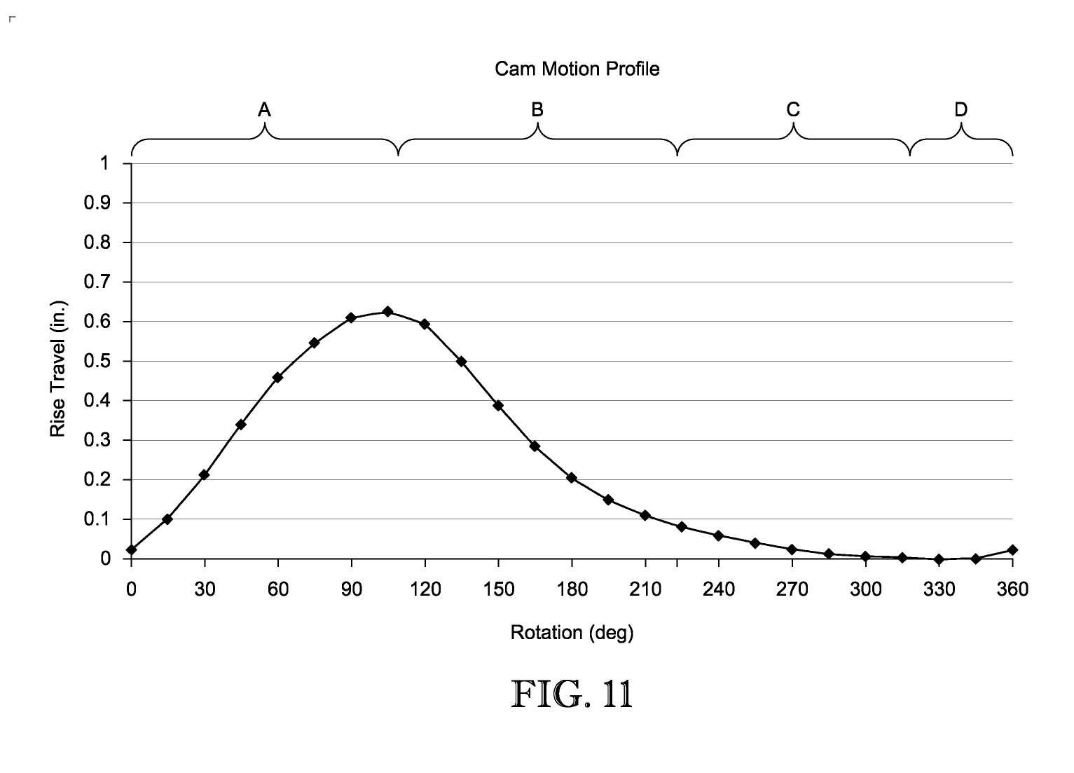

FIG. 11 graphically illustrates the change in elevation of platform 38 as cam 50 rotates through a full revolution. Note, FIG. 11 contains reference lines identifying parts A-D of the raising and lowering cycle. It is clearly visible that the time for platform 38 to shift from its lowest configuration to its highest configuration (here equivalent to degree of rotation as cam 50 is powered by a constant speed motor) is much less than the time for the cam to shift from its highest configuration back to its lowest configuration. Thus, the motion profile of the apparatus follows the breathing patterns of an adult with the “exhale” motion typically taking longer to occur than the “inhale” motion and a slight, but noticeable, pause between cycles to simulate the pause after exhalation and before inhalation of breath. In certain embodiments, the change in platform elevation during a raising and lowering cycle is between about 0.5 to about 1.0 inch, or between about 0.55 to about 0.8 inch, or about 0.625 inch. In certain embodiments, apparatus 10 operates to achieve between 6 to 18 raising and lowering cycles per minute, or between 12 to 14 cycles per minute, thereby simulating the average respiratory patterns of an adult at rest.

Apparatus 10 also delivers consistent motion from cycle to cycle. Unlike mechanisms that are entirely spring operated, the amplitude of the raising and lowering motion does not appreciably change during the course of several raising and lowering cycles. The amplitude and/or period of motion with a spring-operated device would diminish over time until the device stopped. The present invention makes it possible to simulate a regular respiratory rhythm for extended periods of time.

As shown in the drawings, platform 38 is slightly inclined. In certain embodiments, this incline may be between about 0 to about 15 degrees from horizontal. In alternate embodiments according to the present invention, however, platform 38 or recessed surface 23 of upper portion 22 can be configured with structure permitting the user to adjust the degree of incline as desired from between zero degrees to about 15 degrees. The incline feature assists in preventing infant regurgitation and asphyxiation, reducing negative health effects as a result of the infant's weight, preventing plagiocephaly (flattening of the infant's head), assisting with breathing difficulties due to congestion, and improving fluid and mucus drainage. As illustrated in the drawings, platform 38 maintains a generally constant incline throughout the raising and lowering cycle. However, it is within the scope of the present invention for the platform to pivot about a fixed point as it rises and descends.

I claim:

1. An apparatus into which an infant may be placed and operable to simulate the respiratory patterns of an adult, said apparatus comprising:
a bed structure into which an infant may be placed; and
a base assembly including a mechanism for powered raising and lowering of said bed structure between a first lowered position and a second raised position, said mechanism maintaining said bed structure at substantially the same level of inclination during shifting between said first and second positions.

2. The apparatus according to claim 1, wherein said mechanism comprises a cam assembly operably coupled with said bed structure by one or more linkage members.

3. The apparatus according to claim 2, wherein said linkage members are coupled with a shiftable platform upon which said bed structure is received.

4. The apparatus according to claim 3, said linkage members being pivotally coupled to said platform.

5. The apparatus according to claim 2, said cam assembly being operably coupled to an electric motor.

6. The apparatus according to claim 2, said cam assembly comprising a pair of cams mounted to an axle, said axle being rotatably coupled to an electric motor by a drive belt.

7. The apparatus according to claim 6, said cam assembly further comprising a pair of follower bearings, each of said follower bearings coupled with a respective linkage member that is coupled to said bed assembly.

8. The apparatus according to claim 1, said bed structure comprising a detachable bassinet.

9. The apparatus according to claim 1, said mechanism being operable to shift said bed structure from said first lowered position to said second raised position in less time than said mechanism shifts said bed structure from said second raised position back to said first lowered position.

10. The apparatus according to claim 1, said base assembly comprising a fan assembly for moving air from around said bed structure.

11. An infant-supporting apparatus comprising:
a base assembly including—
a platform pivotally, connected to a stationary support through one or more linkage members,
a rotatable cam operably coupled to at least one of said linkage members,
a motor operably coupled with said cam for powered rotation thereof,
said motor, cam, and linkage members being operable to effect shifting of said platform between a first lowered position and a second raised position while maintaining said platform at a generally constant incline throughout said shifting; and
a bed unit received on said base assembly and configured to receive an infant therein, said bed unit being raised and lowered along with said platform during shifting of said platform between said first and second positions.

12. The apparatus according to claim 11, said base assembly comprising a pair of rotatable cams mounted to an axle that is operably coupled with said motor by a drive belt.

13. The apparatus according to claim 11, said motor and cam being operable to shift said platform from said first lowered position to said second raised position in less time than said motor and cam shift said platform from said second raised position back to said first lowered position.

14. The apparatus according to claim 11, said bed unit comprising a bassinet detachably received on said base assembly.

15. The apparatus according to claim 11, said base assembly further comprising a spring mounted between said stationary support and said platform to assist in shifting of said platform between said first lowered position and said second raised position.

16. The apparatus according to claim 11, said base assembly further comprising a fan assembly for moving air from around said bed unit.

17. A method of providing therapeutic motion for an infant comprising the steps of:
placing an infant in an infant-supporting apparatus comprising a mechanism for powered shifting of said apparatus between a first lowered position and a second raised position; and
activating said mechanism to cause said apparatus to oscillate between said first lowered position and said second raised position, wherein the time in which said apparatus shifts from said first lowered position to said second raised position is less than the time in which said apparatus shifts from said second raised position back to said first lowered position.

18. The method according to claim 17, said infant-supporting apparatus comprising a base assembly and a bed unit, said base assembly including a shiftable platform and a stationary support, said mechanism shifting said platform relative to said stationary support upon being activated.

19. The method according to claim 18, said platform being coupled to said support by a plurality of linkages, at least one of said linkages being operably coupled to said mechanism.

20. The method according to claim 19, said mechanism comprising at least one cam operably coupled to an electric motor, said electric motor rotating said at least one cam to effect shifting of said apparatus between said first lowered position and said second raised position.
